(19) United States
(12) Patent Application Publication
(10) Pub. No.: US 2022/0192927 A1
(43) Pub. Date: Jun. 23, 2022

(54) SYSTEM AND METHOD FOR MEDICATION COMPLIANCE AND DRUG ABUSE PREVENTION

(71) Applicant: Nivedita Lakshmi Mehul Sanghavi, San Jose, CA (US)

(72) Inventor: Nivedita Lakshmi Mehul Sanghavi, San Jose, CA (US)

(21) Appl. No.: 17/130,379

(22) Filed: Dec. 22, 2020

(51) Int. Cl. A61J 7/04; A61J 1/14; G16H 40/40; G16H 20/10; G16H 10/60; G16H 40/67; G16H 70/40; G06F 21/31; G06F 3/01; G08B 21/18; G01G 19/52

(52) U.S. Cl. CPC A61J 7/0445; A61J 1/1412; G16H 40/40; G16H 20/10; G16H 10/60; G16H 40/67; G06F 3/0482; G06F 21/31; G06F 3/017; G08B 21/182; G01G 19/52; A61J 2200/74; A61J 2200/30; G16H 70/40

(57) ABSTRACT

A medication compliance and drug abuse prevention device for use as a cap with conventional medication container. The neck of the cap is adapted for mating with the neck of any conventional medication container. The mating of the cap neck with the container neck forms a seal therebetween and the seal can be maintained in a locked or unlocked state as per requirement. The cap has a weighment module configured to measure a mass of a medicinal substance contained inside the container. A control module determines a difference in the mass of the medicinal substance measured before and after the unlocked state and initiate one or more alert actions if the difference in mass of the medicinal substance is found to be less than or more than a prescribed mass of medicinal substance.

### FIELD OF THE INVENTION

The present invention generally relates to the field of medication usage management. More particularly, the present invention relates to a device, system and method which ensures medication compliance and prevents drug abuse.

### BACKGROUND OF THE INVENTION

Medication adherence is defined by the World Health Organization as “the degree to which the person's behavior corresponds with the agreed recommendations from a health care provider.” Though the terms adherence and compliance are synonymously used adherence differs from compliance. Compliance is the extent to which a patient's behavior matches the prescriber's advice. Compliance implies patient's obedience to the physician's authority, whereas adherence signifies that the patient and physician collaborate to improve the patient's health by integrating the physician's medical opinion and the patient's lifestyle, values and preferences for care.

Medications are taken for varying reasons/to address varying health concerns. Some medications are taken to treat an illness while some medications are taken to keep vital parameters within low risk ranges. Some medications are also taken as nutritional supplements. Medications are also taken for many other reasons. Different medications are taken for different duration of time. Some are taken for a short duration of time, some for a long duration while some medications are to be taken for life time. In case of a viral infection, the patient may be prescribed a medication for a very short duration. In case of a complicated disease, the patient/user may be prescribed a medication for a longer duration. In case of life-threatening diseases, patients/users are prescribed medications for life time. In case of medications for longer duration or life time use, patients might receive a prescription for a specific period after which the prescription will expire. Patients will have to renew prescription to continue use of medication which patients/users tend to forget. Many patients/users take multiple medications in a day to take care of multiple health issues or for other reasons. Different medications are to be taken at different times of the day and in different quantities. Some medications are to be taken only once a day while some are to be taken more than once a day. For patients who must take three, four or even five dosages of different medications daily, the prescribed course of medical treatment can easily become confusing. For medications to work efficiently, they are required to be taken in the manner prescribed by a health care professional. However, patients/user, most of the time, fail to follow their physician's dosage instructions—some out of carelessness and some forget to take their medication on time, some are unpunctual in taking a dose, or forget entirely to take a dose and/or take a second dose too soon and in some cases fail to understand the manner in which the medications are to be taken due to unfamiliarity with a new medication schedule. Being occupied with an activity such as official work, school, or physical activities and simply forgetting to take the medications on time also lead to medication non-compliance. It can also be particularly difficult for children to remember to take their medications on time, especially if any doses are required to be taken while the child is away from home, such as during school or while at day care.

In cases where multiple medications are to be taken at different times of the day, sometimes it becomes a challenging task to keep a track of dosage amount and dosing times. Each medication has its own appropriate dosing timetable so puzzlement between medications can lead to threatening under or over dosing circumstances. The potential risk caused by drug interactions may demand a time gap between some kinds of medications. Forgetfulness to self-administer the medication prescribed by a health care professional at the right intervals and right dosages by patients/users has long been found to be one of the biggest hurdles for health care professionals in determining the effectiveness of prescriptions. The more frequently a medication must be taken, the more likely the patient's compliance rate will drop. Inadvertently taking a medication twice, missing a dose of medication, and/or harmful interaction between medications are all significant reasons of people's visit to emergency rooms.

Medication non-compliance is a serious issue in the health care sector. Medication non-compliance leads to poor clinical results. During initial course of start of medication, patients/users do their best to make sure they take medications in the prescribed manner. However, in due course, due to numerous different factors discussed above, patients/users fail to take medications in the prescribed manner. Non-compliance to a prescribed schedule can cause adverse effects, such as unnecessary aggravations, repeating symptoms, additional doses of emergency treatment medication, and/or hospital emergency room visits. For example, a kidney failure patient is prescribed an average of ten medications, and some are prescribed as many as twenty different types of medication. With half of these medications taken three or four times per day, this amounts to multiple medications per day, all needing to be systematized to be taken at specific times and following specific instructions, such as taken with food, with water, empty stomach etc. Lack of medication compliance is a primary reason for patients being re-hospitalized after discharge from the hospital and medication related morbidity. Medication non-compliance leads to seniors needing extra and often more expensive care, such as assisted living provisions or nursing homes. Complying with a schedule can thus help to maintain a patient's health, help reduce situations of emergency medication administration, and/or help reduce health care expenses by requiring lesser emergency hospital visits or other health care professional's consultation fees.

In addition to medication non-compliance, another serious issue in the health care sector is medicinal drug abuse. The prevalence of medicinal drug use and abuse worldwide is a well known fact. Medicinal drug use and abuse have become grave public policy issues and are having an impact on all class of society with its obvious social and medical outcomes. Medication non-compliance is unintentional and as discussed above, most of the time it is due to forgetfulness or carelessness. Unfortunately, some people intentionally don't follow the dosage instructions and consume more dose than that was recommended by a health care professional for the sole reason of getting intoxicated. There are numerous different factors that lead to medicinal drug abuse. The properties of some medications make people addictive. So some medications which are extremely effective for improving life quality of patients attract drug abusers because of their abuse potential. For example, some medications that can control severe chronic pain have abuse potential and thus attract drug abusers. Sometimes a person has the tendency to become an addict. A person who is emotionally down thinks he can feel good by getting himself intoxicated which in due course becomes a habit. People suffering financial crisis, other hardships and personal circumstances or out of peer pressure get involved in medicinal drug abuse. Everyone does not have access to illegal street drugs. Then they start taking certain prescribed medications of a family member, of a friend or of self in higher doses for the sole purpose of getting intoxicated and thereupon allege that the medications are lost, misplaced or stolen.

Medication non-compliance and medicinal drug abuse are, thus, a serious problem throughout the world. Because of such issues and others, it is extremely important that today's technology come up with befitting clear command on when a particular medication is needed to be taken, how the medication is to be taken and who can have access to the medication. Hence there is a need for medication dispensers and reminders and a device that integrate reminding, alerting and dispensing functions that can help patients/user manage their medications and diminish the risk that a patient/user will forget taking his medication, maintain the correct schedule of a medication or multiple medications, control the potential for a patient/user to take a medication inadvertently more than the recommended dose, alert patient/user that the medication is nearing end and/or restrict medicinal drug abuse by restricting access to the medication/medications.

There are medication organizers which assist in tracking the medications needed to be taken. The organizers are available in different styles. For example, there are daily medicine containers where one can store medicines for each day of the week or they even rely on someone else to tell them when to take their medication. This might be effective to some extent but does not completely do away with the possibility of the patient/user becoming confused or simply forgetful in addition to the lack of an infallible compliance record. Such daily medicine containers require effort on the part of the user and the user needs to keep a track of the medications taken/to be taken manually which leads to medication non-compliance. It even becomes difficult for caregivers or family members to obviate medication non-compliance.

Although, different devices and systems have been developed to prevent medication non-compliance, they have notable flaws. Medication is usually supplied in a container on which detailed information regarding the medication is provided. The detailed information furnishes significant data as to the distinguishing features of the medication and the dosing instructions for that medication. Having access to that data is important to the patient/user. Patients/users generally transfer medication from its original dispensary packaging to other containers often when they have several medications to take. For example, the patient/user might transfer several different medications that they expect taking over the course of an entire week to a container having sub compartments for each day of the week. Separating medications from its original dispensary packaging to other containers can be risky. This can confuse the patient/user as they no longer will have access to the data and this might lead to over dosing and under dosing situations. It can also happen that the medications become damp and lose their properties. Another very alarming disadvantage about transferring medications from original container is that drug abusers can steal medications without the knowledge of the intended user of the medication. There is no such device that can prevent drug abusers from using someone else's prescribed medication. Also, no such device exists which can prevent a patient from taking more than the prescribed amount of drug.

It is, therefore, highly desirable to have a device that helps is enforcing medication compliance and effectively prevents drug abuse.

### OBJECTS OF THE INVENTION

It is, therefore, an object of the present invention is to provide a system and method for medication compliance and drug abuse prevention.

Another object of the present invention is to provide a device that can be fitted to any standard/conventional medication packaging.

Another object of the present invention is to provide a device that can be fitted to the original dispensary packaging of medications without having to transfer the medications from its original dispensary packaging to another dispenser.

Another object of the present invention is to provide a device that can detect when a medication dispenser has been fitted to it, removed, opened or otherwise tampered with and report such activity to the user of the device.

Another object of the present invention is to provide a system and a method that addresses medication non-compliance concern.

Another object of the present invention is to provide a system and a method that can prevent potential medicinal drug abuse.

Another object of the present invention is to provide a device that can remind a user of the device to take a medication.

Another object of the present invention is to provide a device that can notify a user of the device to refill medications.

Another object of the present invention is to provide a device that can prevent a user of the device from taking accidental overdose.

Another object of the present invention is to provide a device that helps the user of the device to take the correct medication on the right time with the correct medication and alert the user when wrong medication is selected.

Another object of the present invention is to provide a device which does not give medication access to the user of the device if the user had already taken the required dose.

Another object of the present invention is to provide a device which does not allow unauthorized access to medication.

Another object of the present invention is to provide a device that detects tampering attempt and takes appropriate action.

Another object of the present invention is to provide a device that make audible beeps, or send text messages to remind/notify/alert a user of the device about an action to be taken or an activity that has occurred.

Another object of the present invention is to provide a device that provides visual information to a user of the device.

Another object of the present invention is to provide a system and a method that can interact with one or more third parties, such as pharmacies, healthcare professionals, and healthcare products suppliers through a computer network.

Other objects and further scope of applicability of the present invention will become apparent from the detailed description given hereinafter. It should be understood, however, that the detailed description and specific examples, while indicating preferred embodiments of the invention, are given by way of illustration only, since various changes and modifications within the spirit and scope of the invention will become apparent to those skilled in the art from this detailed description.

### SUMMARY OF THE INVENTION

The following presents a simplified summary in order to provide a basic understanding of some aspects of the disclosed invention. This summary is not an extensive overview, and it is not intended to identify key/critical elements or to delineate the scope thereof. Its sole purpose is to present some concepts in a simplified form as a prelude to the more detailed description that is presented later.

The present invention is directed to a medication compliance and drug abuse prevention device, system and method. The medication compliance and drug abuse prevention system comprising one or more medication compliance caps, one or more computing devices and a remote server either retrieves all the medication information applicable to a user or such information is fed to the system. The system takes help of this information and configures the medication compliance cap accordingly to ensure medication compliance and abuse prevention. The medication compliance cap replaces the original cap of a medicine container for functioning of the present invention. The medication compliance cap comprises a main body having a top end, a bottom end and a side wall extending between the top end and the bottom end. The neck of the cap disposed at the top end is adapted for mating with the neck of a container to which the medication compliance cap is being attached. The mating of the cap neck with the container neck forms a seal therebetween. A locking mechanism disposed at the cap neck enables the seal to be maintained in a locked state or in an unlocked state as per requirement. The medication compliance cap further comprises a control unit having a processor which executes one or more instructions to maintain the locked and unlocked state as per a medication schedule/instruction or as per a successful user authentication process. A weighment module disposed inside the cap compartment is configured to measure, when the container is kept in an inverted position, on every occasion, the mass of a medicinal substance contained inside the container's internal space before and after the seal is unlocked. The control module determines a difference in the mass of the medicinal substance measured before and after the unlock state to find if any anomaly occurs in terms of the difference in mass of the medication not matching a recommended dose of prescribed medication for which the unlock state was executed. One or more alert actions or preventive/corrective actions are initiated if such an anomaly is found. A companion application installed on a computing device communicatively connected to the medication compliance cap provides, on its own or in association with a remote server, a user interface on the computing device. Each medicine container requires a separate medication compliance cap and the user interface can be used to associate all such medication compliance caps with a user identity or user account.

To the accomplishment of the foregoing and related ends, certain illustrative aspects of the disclosed invention are described herein in connection with the following description and the annexed drawings. These aspects are indicative, however, of but a few of the various ways in which the principles disclosed herein can be employed and is intended to include all such aspects and their equivalents. Other advantages and novel features will become apparent from the following detailed description when considered in conjunction with the drawings.

### BRIEF DESCRIPTION OF THE DRAWINGS

The accompanying drawings, which are incorporated herein and form a part of the specification, illustrate the present invention and, together with the description, further serve to explain the principles of the invention and to enable a person skilled in the relevant art to make and use the invention. The present invention will become more fully understood from the detailed description given herein below and the accompanying drawings which are given by way of illustration only, and thus are not limitative of the present invention, and wherein:

FIG. 1A illustrates a conventional pill/tablet/capsule container;

FIG. 1B illustrates a conventional pill/tablet/capsule container having threaded cap;

FIG. 1C illustrates a conventional pill/tablet/capsule container having a press-fit or snap-on type of cap;

FIGS. 2A-2C illustrate first embodiment of the medication compliance and abuse prevention cap of the present invention;

FIGS. 3A-3B illustrate second embodiment of the medication compliance and abuse prevention cap of the present invention;

FIG. 4 illustrates a conventional container with threaded neck before being attached to the first embodiment of the medication compliance and abuse prevention cap of the present invention;

FIG. 5 illustrates a conventional container with threaded neck attached to the first embodiment of the medication compliance and abuse prevention cap of the present invention;

FIG. 6 illustrates a conventional container suitable for snap-on or push-fit type of cap before being attached to the second embodiment of the medication compliance and abuse prevention cap of the present invention;

FIG. 7 illustrates a conventional container suitable for snap-on or push-fit type of cap attached to the second embodiment of the medication compliance and abuse prevention cap of the present invention;

FIGS. 8A and 8B illustrate conventional containers holding pills/tablets/capsules/medication substance inside and fitted with the medication compliance and abuse prevention cap in accordance with the first and second embodiments of the present invention;

FIG. 9 illustrates a general architecture of the medication compliance and abuse prevention cap in accordance with the first and second embodiments of the present invention;

FIG. 10 illustrates a general architecture of a computing device that operates in accordance with an embodiment of the present invention;

FIG. 11 illustrates a general architecture of a medication compliance and abuse prevention system that operates in accordance with an embodiment of the present invention;

FIGS. 12A-12B illustrate non-limiting exemplary screenshots of Graphical User Interface (GUI) or user interface provided by the present invention showing logon/signup screens in accordance with an embodiment of the present invention;

FIG. 13A illustrates a non-limiting exemplary screenshot of Graphical User Interface (GUI) or user interface provided by the present invention showing steps for associating a medication compliance cap with a user account in accordance with an embodiment of the present invention;

FIG. 13B illustrates a non-limiting exemplary screenshot of Graphical User Interface (GUI) or user interface provided by the present invention showing introductory information in accordance with an embodiment of the present invention;

FIGS. 14A-14B illustrate non-limiting exemplary screenshots of Graphical User Interface (GUI) or user interface provided by the present invention showing information on medication and overall status of associated medication compliance and abuse prevention caps in accordance with an embodiment of the present invention;

FIGS. 15A-15B illustrate non-limiting exemplary screenshots of Graphical User Interface (GUI) or user interface provided by the present invention showing setting of medication sequence and status of the medication compliance and abuse prevention caps associated with a user in accordance with an embodiment of the present invention;

FIGS. 16A-16C illustrate non-limiting exemplary screenshots of Graphical User Interface (GUI) or user interface provided by the present invention showing controls for settings and notification/alert for medication associated with medication compliance and abuse prevention caps in accordance with an embodiment of the present invention; and

FIG. 17 illustrates a flowchart depicting the general steps associated with the method for medication compliance and abuse prevention system in accordance with an embodiment of the present invention.

The present invention will now be described with reference to the accompanying drawings. In the drawings, generally, like reference numbers indicate identical or functionally similar elements. Additionally, generally, the left-most digit(s) of a reference number identifies the drawing in which the reference number first appears.

### DETAILED DESCRIPTION OF THE INVENTION

The following detailed description of the present invention refers to the accompanying drawings that illustrate exemplary embodiments consistent with this invention. Other embodiments are possible, and modifications can be made to the embodiments within the spirit and scope of the invention. Therefore, the detailed description is not meant to limit the invention. Rather, the scope of the invention is defined by the appended claims.

It would be apparent to one of skill in the art that the present invention, as described below, can be implemented in many different embodiments of software, hardware, firmware, and/or the entities illustrated in the figures. Any actual software code with the specialized control of hardware to implement the present invention is not limiting of the present invention. Thus, the operational behavior of the present invention will be described with the understanding that modifications and variations of the embodiments are possible, given the level of detail presented herein.

Reference now will be made in detail to embodiments of the invention, one or more examples of which are illustrated in the drawings. Each example is provided by way of explanation of the invention, not limitation of the invention. In fact, it will be apparent to those skilled in the art that various modifications and variations can be made in the present invention without departing from the scope or spirit of the invention. For instance, features illustrated or described as part of one embodiment can be used with another embodiment to yield a still further embodiment. Thus, it is intended that the present invention covers such modifications and variations as come within the scope of the appended claims and their equivalents.

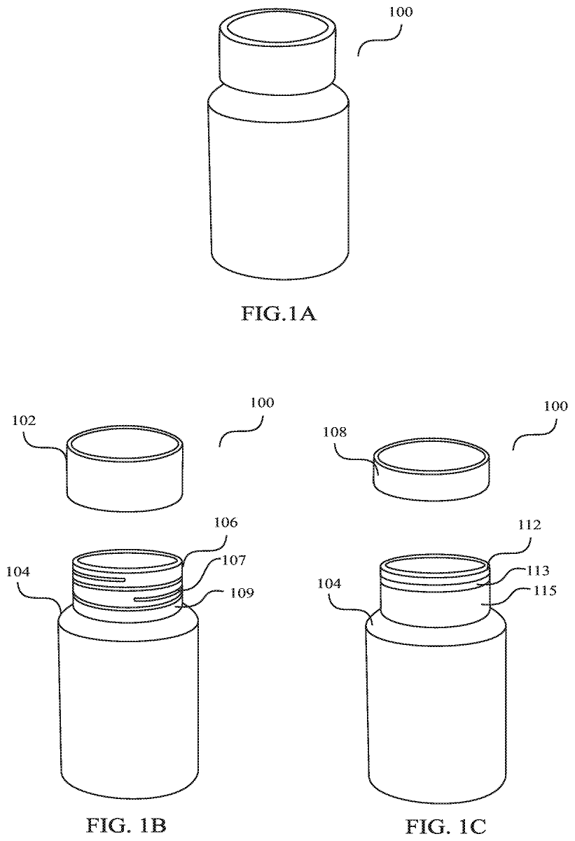

FIGS. 1A-1C illustrate a conventional container/bottle 100 which is widely used for storing solid medicinal substance such as pills/tablets, capsules or for storing any liquid medicinal substance. There are two basic types of capping used for such conventional containers—a screw-on or threaded type as shown in FIG. 1B and a snap-on type as shown in FIG. 1C. The neck portion 106 of the container body 104 is provided with neck threads 107 in threaded type of container as shown in FIG. 1B. The threads of the cap 102 engage with the threads 107 of container neck to close the container. In snap-on type container, as shown in FIG. 1C, the cap 108 can close the container by engaging with the annular ring 113 provided on the neck 112 of the container body 104 when pushed/pressed.

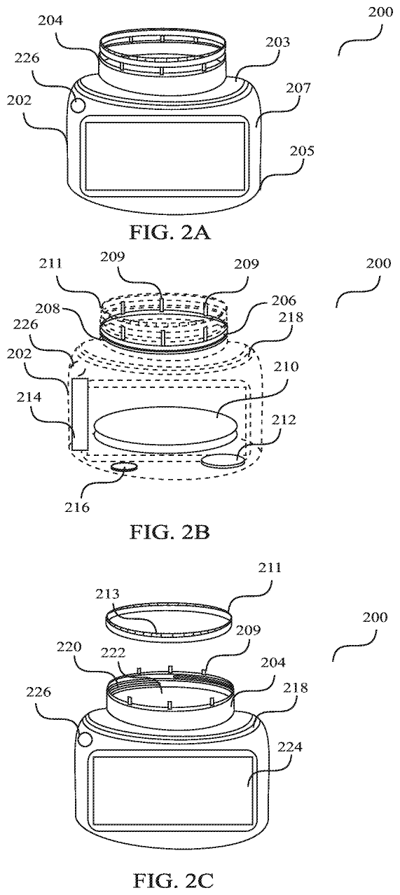

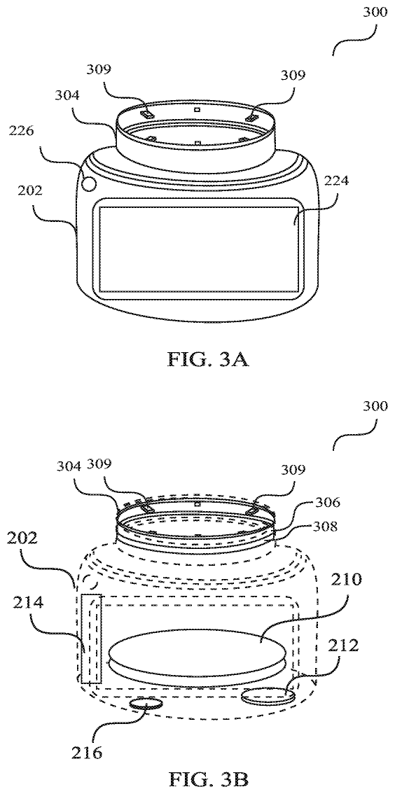

The present invention is a device, system and method for ensuring medication compliance and preventing drug abuse. As a part of the system of the present invention, a medication compliance and drug abuse prevention device is provided which can work alone or in association with one or more remotely connected additional computing devices configured to help the device and users of the device to ensure medication compliance and drug abuse prevention. The medication compliance and drug abuse prevention device of the present invention can be used as a cap for any conventional medication container/bottle 100 in place of the original cap of the container. The same medication compliance and drug abuse prevention cap can be reused with another medication container after it is used with a first container or bottle. For each container/bottle of orally taken medicine, a user will need one medication compliance and drug abuse prevention cap. The terms medicine and medication are used herein alternatively and interchangeably. The cap of the present invention can be made to fit with any standard medicine container or bottle. For screw-on or threaded type of conventional container, the cap of the present invention can be made as a screw-on type cap 200 as shown in FIGS. 2A-2C. For snap-on type of conventional containers, the cap of the present invention can be made as a snap-on compatible cap 300 as shown in FIG. 3. In some embodiments, a compatible container or a bottle can also be provided with the medication compliance and drug abuse prevention cap to which the medicinal substance from an original conventional container of the medicine can be transferred before use. The term medication compliance and drug abuse prevention system is hereinafter also referred to as MEDCOMAP system. All the embodiments (200, 300, for example) of the medication compliance and drug abuse prevention cap are alternatively and collectively referred to as MEDCOMAP cap or medication compliance cap.

FIGS. 2A-2C illustrate a first embodiment of the medication compliance and drug abuse prevention device/cap of the present invention which is a screw-on type cap. The MEDCOMAP cap/medication compliance cap 200 comprises a main body 202, a cap neck 204, a locking mechanism 206, a weighment module 210 and a control module 214. The main body 202 has a top end 203, a bottom end 205 and a sidewall 207 extending between the top end 203 and the bottom end 205. The sidewall 207 defines a cap internal compartment/space/cavity 222 as shown in FIG. 2C which is closed at the bottom end 205 and open at the top end 203. The cap neck 204 is disposed at the top end 203 and it includes cap threads 220 similar to the threads of a conventional container's cap.

Reference to FIG. 2B, the locking mechanism 206 comprises an actuator 208, one or more teeth or latches 209 movably connected to the actuator 208 and a collar 211. An example of the actuator 208 can be a solenoid actuator which, when energized or de-energized, is able to push or pull the teeth/latches 209 upward or downward or rotate the teeth/latches in a desired direction to engage with the receiving slots/openings/grooves 213 provided in the collar 211 shown in FIG. 2C.

The medication compliance cap 200 further comprises a power source 212, a speaker 216, one or more light sources 218 and a cap user interface 224 as shown in FIGS. 2B and 2C. The power source 212 can be a battery which is required to supply required electrical power to the components of the Medication compliance cap. The speaker 216 is configured to give audible alerts and the light sources 218 give visual alerts.

A snap-on type of medication compliance cap 300, which is the second embodiment of the present invention, is illustrated in FIGS. 3A and 3B. Medication compliance cap 300 is similar to MEDCOMAP cap 200 in structure and function except with the difference in the cap neck and locking mechanism. In medication compliance cap 300, the cap neck 304 does not have threads. Instead, the cap neck 304 is so configured that it fits snugly around a snap-on type conventional container's neck when pushed down. The locking mechanism 306 of medication compliance cap 300 comprises an actuator 308 and one or more teeth or latches 309. The teeth 309 are disposed radially around the inside periphery of the cap neck 304. The actuator 308 makes the teeth 309 move toward the center of the cap neck 304 when required.

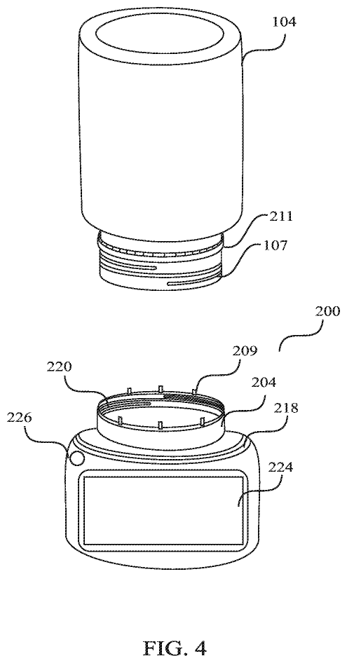

In case of the embodiment of the present invention in which the medication compliance cap is provided without any accompanying container, the original cap of a conventional medicine container, in which medicine/medicinal substance is bought/supplied, is required to be replaced with the medication compliance cap by the user before start of medication. FIG. 4 shows a screw-on type medication compliance cap 200 before being fitted to a conventional screw-on type medicine container body 104. Here, as shown in FIG. 4, the collar 211 of the medication compliance cap 200 is required to be attached to the unthreaded portion 109 of the container neck 106 located behind the container neck threads 107 (the container neck 106, the container neck threads 107 and the unthreaded portion 109 are shown in FIG. 1B). In a preferred embodiment, the collar 211 is made of flexible material so that the collar 211 can be pushed through the container neck 106. The collar 211 slides over the container neck threads 107 and fits in the unthreaded portion 109 when pushed or pressed. In another embodiment, the collar 211 is made split type so that the collar 211 can be easily installed over the unthreaded portion 109. In either case, once the collar 211 is installed in the container neck it cannot be taken out without breaking/tearing the collar 211. Therefore, the collar 211 can be made available as a separately purchasable product in the market for users.

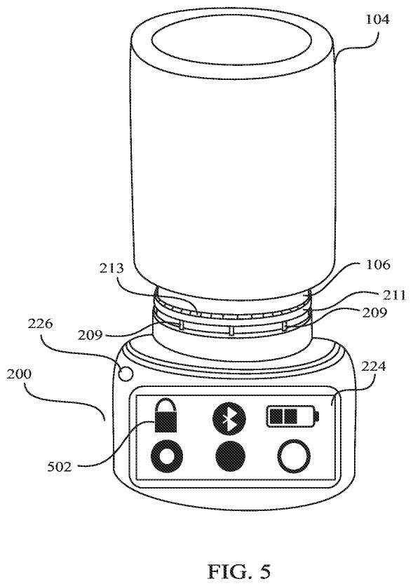

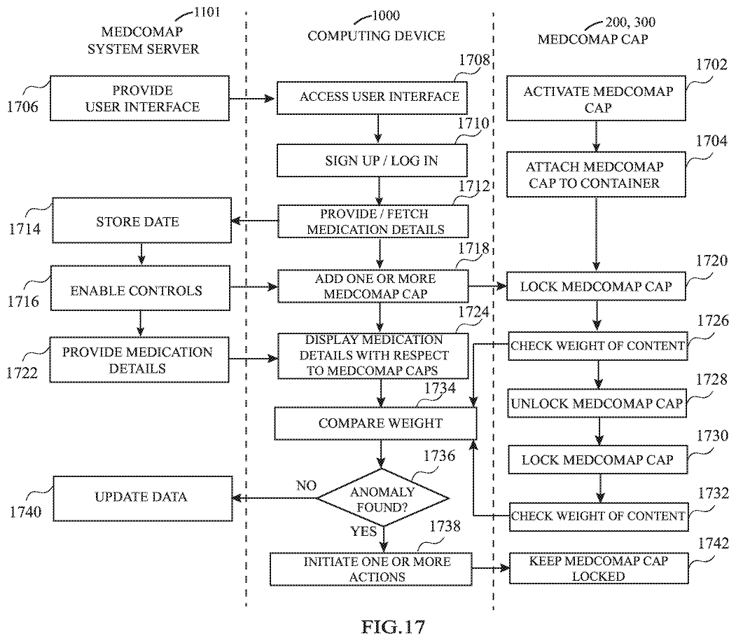

In one embodiment, reference to FIGS. 2A and 3A, a power button 226 is disposed on the medication compliance cap which can be pressed manually to turn on/activate the medication compliance cap as in step 1702 of FIG. 17. Once activated, the cap user interface 224 presents one or more information on the status of the medication compliance cap and one or more options for controlling the functions of the medication compliance cap. In a preferred embodiment, the locking mechanism 206 is so configured that the teeth 209 remain in extended position as shown in FIG. 4 when the medication compliance cap is turned off or the actuator 208 is not energized. The medication compliance cap 200 is then screwed onto the container neck 106, by threaded engagement of the container neck threads 107 with the cap threads 220, while the one or more teeth 209 are retained in a retracted position by energizing the actuator 208. The medication compliance cap 200 has to be rotatably adjusted till it comes close to the collar 211 as in step 1704 of FIG. 17. The cap neck 204 thus mates with the container neck 106 and forms a seal therewith. The medication compliance cap 200 can then be locked by the action of the engagement of the teeth 209 with the receiving slots 213 disposed on the collar 211 as in step 1720 of FIG. 17. FIG. 5 illustrates the medication compliance cap 200 in locked position with the container body 104. The in-built cap user interface 224 indicates the status of the medication compliance cap 200 as locked in FIG. 5 by displaying an icon 502.

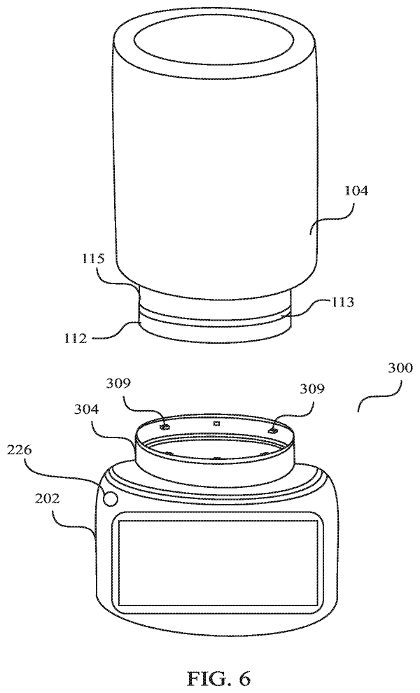

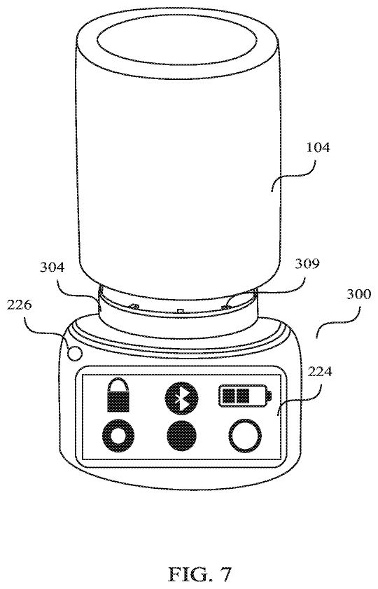

FIG. 6 displays the second embodiment of the present invention, the medication compliance cap 300, before being attached to a snap-on type conventional container. In a preferred embodiment, as in medication compliance cap 200, the locking mechanism 306 of medication compliance cap 300 is so configured that the teeth 309 remain in radially extended position when the medication compliance cap 300 is turned off or the actuator 308 is de-energized. The medication compliance cap 300 can be pressed over the container neck 112 (container neck 112 is shown in FIG. 1C) for attachment so that the cap neck 304 mates with the container neck 112 and forms a seal therewith. The medication compliance cap 300 can be turned on/activated by manually pressing power button 226 as in step 1702. Once attached, as shown in FIG. 7, the teeth 309 are made to extend to grip the container neck 112, preferably around the unthreaded area 115, located behind the annular ring 113 thereby locking the medication compliance cap 300 with the container neck 112 as in step 1720 of FIG. 17. The fact that the locking mechanism of both the embodiments 200 and 300 maintains the teeth in extended position in de-energized condition ensures that the medication compliance cap remains locked even if someone turns it off or the battery is removed. This feature helps in preventing drug abuse as the lock of the medication compliance cap cannot be compromised by switching off the power.

In an attached position, the cap neck of the medication compliance cap, in association with the container neck, defines a passage between the cap internal compartment and the container internal space.

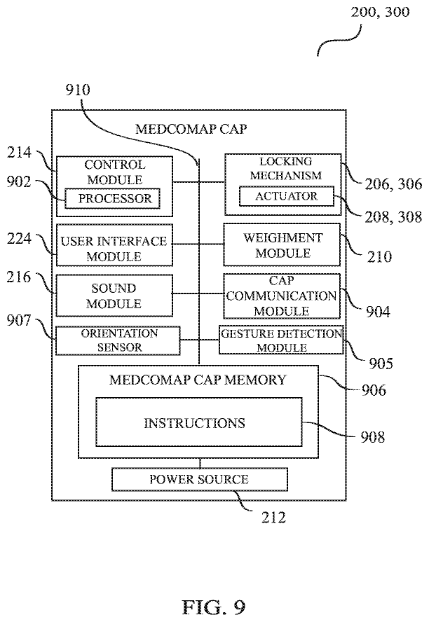

FIG. 9 is an exemplary block diagram for the various components of the medication compliance cap 200 or 300. The control module 214 comprises one or more physical processors 902. The processor 902 can be provided by a general purpose microprocessor or by a specialized microprocessor (e.g., an ASIC). A bus 910 or other similar communication mechanism is provided in the medication compliance cap for communicating information, and for supplying electrical power to the components of the medication compliance cap. The medication compliance cap 200 or 300 further comprises a cap communication module 904 and a memory 906. The memory 906 is coupled to the bus 910 for storing information and instructions 908 to be executed by the processor 902, including information and instructions for performing the techniques described below, for example. This memory may also be used for storing variables or other intermediate information during execution of instructions to be executed by processor 902. A storage device can also be provided for storing information and instructions. Common forms of storage devices include, for example, a flash memory, a USB memory card, or any other medium from which a computer can read. Storage device and memory are both examples of computer readable media.

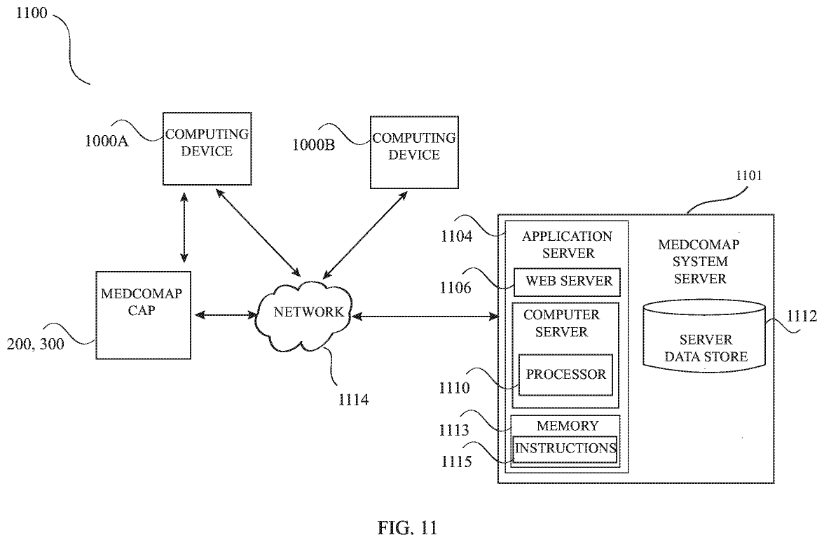

The cap communication module 904 is configured to enable communication of the medication compliance cap with other devices. Such communication can be performed wirelessly (by radio frequency or infrared communication, for example), or by wire. Communication module 904 is hardware and software configured to transmit data to and from the medication compliance cap. For example, the communication module 904 is configured to transmit data from the medication compliance cap to one or more computing devices 1000A (referred to as first computing device) and 1000B (referred to as second computing device) and to a MEDCOMAP system server 1101 as shown in FIG. 11. The control module 214 or the processor 902 may receive data from the computing devices 1000A or 1000B and from MEDCOMAP system server 1101 through the communication module 904. Hence, communication module 904 is configured to facilitate bidirectional transmission of data. The communication module 904 can include a wireless module and such a wireless module can be a Wi-Fi module. Alternatively, the wireless module may be a Bluetooth module, a CDMA module, or any other communication module that enables a wireless communication link for the bidirectional flow of data between the devices wirelessly.

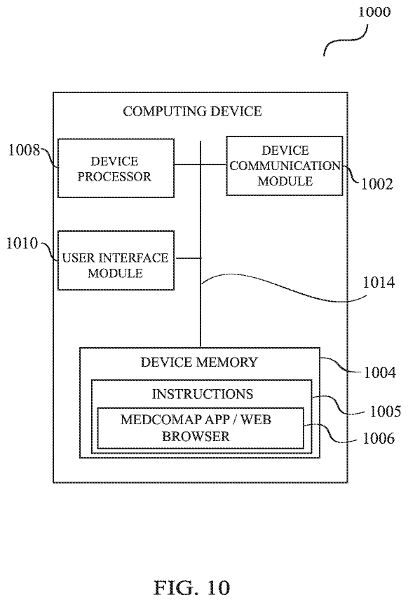

FIG. 10 illustrates computing device 1000 that is representative of any system or collection of systems in which the various applications, services, scenarios, and processes disclosed herein may be implemented. Examples of systems or device form factors in which the computing device 1000 can be implemented include mobile devices such as smartphones, tablets, laptops, smart watches, smart glasses and other wearables, and smart home appliances or home security devices or dedicated base stations. Mobile device 1000 has been used as an example of one such device to describe the steps of the invention here.

Computing device 1000 may be implemented as a single apparatus, system, or device or may be implemented in a distributed manner as multiple apparatuses, systems, or devices. Reference to FIG. 10, computing device 1000 includes, but is not limited to, device communication module 1002, device memory 1004, device processor 1008 and user interface module 1010. The device processor 1008 is operatively coupled with device memory 1004, device communication module 1002, and user interface module 1010 through a communication bus 1014. The device memory includes one or more set of instructions 1005 including application 1006. The application or “app” is a computer program that may be downloaded and installed on computing devices using methods known in the art. It can also be a set of instructions programmed on the hardware of the computing device or a pre-loaded computer program or firmware or any such native application supplied with the computing device. Hereinafter, the app or mobile app/native application of the present invention and/or the user interface of the invention presented through a web browser by the medication compliance and drug abuse prevention system 1100 (system 1100 is illustrated in FIG. 11) are referred to as MEDCOMAP app.

Device processor 1008 loads and executes instructions of MEDCOMAP app 1006 from device memory 1004. When executed by device processor 1008, for medication compliance and abuse prevention, instructions direct device processor 1008 to operate as described herein for at least the various processes, operational scenarios, and sequences discussed in the following implementations. Computing device 1000 may optionally include additional devices, features, or functionality not discussed for purposes of brevity.

Referring still to FIG. 10, device processor 1008 may comprise a micro-processor and other circuitry that retrieves and executes computer readable program instructions 1005 from device memory 1004. Device processor 1008 may be implemented within a single processing device, but may also be distributed across multiple processing devices or sub-systems that cooperate in executing program instructions 1005. Examples of device processor 1008 include general purpose central processing units, application specific processors, and logic devices, as well as any other type of processing device, combinations, or variations thereof.

Device memory 1004 may comprise any computer readable storage media readable by device processor 1008 and capable of storing instructions 1005 including MEDCOMAP app and web browser. Device memory 1004 may include volatile and nonvolatile, removable and non-removable media implemented in any method or technology for storage of information, such as computer readable instructions 1005, data structures, program modules, or other data. Examples of storage media include random access memory, read only memory, magnetic disks, optical disks, flash memory, virtual memory and non-virtual memory, magnetic cassettes, magnetic tape, magnetic disk storage or other magnetic storage devices, or any other suitable storage media.

In particular, the program instructions may include various components or modules that cooperate or otherwise interact to carry out the various processes and operational scenarios described herein. The various components or modules may be embodied in compiled or interpreted instructions, or in some other variation or combination of instructions. The various components or modules may be executed in a synchronous or asynchronous manner, serially or in parallel or in accordance with any other suitable execution paradigm, variation, or combination thereof. Instructions 1005 may include additional processes, programs, or components, such as operating system software, virtual machine software, or other application software, in addition to or that include MEDCOMAP app. Instructions 1005 may also comprise firmware or some other form of machine-readable processing instructions executable by device processor 1008.

In general, instructions 1005 may, when loaded into processor 1008 and executed, transform a suitable apparatus, system, or device (of which computing device 1000 is representative) overall from a general-purpose computing system into a special-purpose computing system customized to facilitate medication compliance and drug abuse prevention.

Device communication module 1002 may include communication connections and devices that allow for communication with other computing systems (devices shown in FIG. 11, for example) over communication networks (network 1114 as shown in FIG. 11, for example). Examples of connections and devices that together allow for inter-system communication may include network interface cards, antennas, power amplifiers, RF circuitry, transceivers, and other communication circuitry.

User interface module 1010 may include a keyboard, a mouse, a voice input device, a touch input device for receiving a touch gesture from a user, a motion input device for detecting non-touch gestures and other motions by a user, and other comparable input devices and associated processing elements capable of receiving user input from a user. Output devices such as a display, speakers, haptic devices, and other types of output devices may also be included in user interface module 1010. In some cases, the input and output devices may be combined in a single device, such as a display capable of displaying images and receiving touch gestures.

User interface module 1010 may also include associated user interface software executable by device processor 1008 in support of the various user input and output devices discussed above. Separately or in conjunction with each other and other hardware and software elements, the user interface software and user interface devices may support a graphical user interface, a natural user interface, or any other type of user interface.

FIG. 11 illustrates computing systems and network environments that may be used to implement aspects of the disclosure. The medication compliance and drug abuse prevention (MEDCOMAP) system 1100 comprises one or more medication compliance caps 200 or 300, one or more computing devices 1000A and 1000B (1000A and 1000B being computing devices capable of performing at least the aforementioned functions) and a MEDCOMAP system server 1101, all communicably connected with each other through a network 1114. The MEDCOMAP system server 1101 may have a processor 1110 for controlling overall operation of the server 1101 and its associated components. The MEDCOMAP system server 1101 may correspond to any of multiple systems or devices, configured as described herein for medication compliance and drug abuse prevention based on information received from the medication compliance cap, the computing devices and from any third party external server (pharmaceutical companies and hospitals, for example) or from user fed data.

The MEDCOMAP system server 1101 may operate in a networked environment 1100 supporting connections to one or more remote computing devices, such as 1000A and 1000B etc. The network connections depicted in FIG. 11 include a local area network (LAN), wide area network (WAN), and a wireless telecommunications network, but may also include other networks. When used in a LAN networking environment, the MEDCOMAP system server 1101 may be connected to the LAN through a network interface or adapter. When used in a WAN networking environment, the system server 1101 may include a modem or other means for establishing communications over the WAN. When used in a wireless telecommunications network, the system server 1101 may include one or more transceivers, digital signal processors, and additional circuitry and software for communicating with wireless computing devices 1000A, 1000B.

It will be appreciated that the network connections shown are illustrative and other means of establishing a communications link between the computers may be used. The existence of any of various network protocols such as TCP/IP, Ethernet, FTP, HTTP and the like, and of various wireless communication technologies such as GSM, CDMA, WiFi, and WiMAX, is presumed, and the various computing devices and the Medication compliance cap described herein may be configured to communicate using any of these network protocols or technologies.

Although, only two computing devices and one medication compliance cap are illustrated in FIG. 11, it is to be understood that there can be a plurality of computing devices and medication compliance caps connected to the MEDCOMAP system server 1101. The MEDCOMAP system server 1101 communicates with the computing devices over the network 1114 to present a user interface for the medication compliance and drug abuse prevention system of the present invention. The user interface of the medication compliance and drug abuse prevention system of the present invention can be presented on the computing device through a web browser or through the native mobile application MEDCOMAP app communicating with the MEDCOMAP system server 1101. The graphical user interface (GUI) or user interface provided by the medication compliance and drug abuse prevention system 1100 on the computing devices through a web browser or through the mobile app may be utilized by the users for signing up, logging in, submitting medication information, accessing medication, giving access to caregivers, receiving/making audio visual alerts, getting real time updates and notifications etc.

The MEDCOMAP system server 1101, as shown in FIG. 11, includes an application server or executing unit 1104, a web server 1106, a processor 1110, a server data store 1112, an application memory 1113 having computer readable instructions 1115. The application server 1104 supports operation of at least one service. Physically, the application server 1104 may be an independent device residing outside the MEDCOMAP system server 1101, or integrated in the MEDCOMAP system server 1101 as shown in FIG. 11, which is not limited by this embodiment in any manner.

The MEDCOMAP system server 1101 performs a portion or all of the processing steps of medication compliance and drug abuse prevention described herein in response to the processor 1110 executing one or more sequences of one or more instructions 1115 contained in the application memory 1113. The term “computer readable medium” as used herein refers to any medium that participates in providing instructions to the processors for execution. A computer readable medium may take many forms, including but not limited to, non-volatile media, volatile media, and transmission media. Non-volatile media includes, for example, solid state drive, optical, magnetic disks, and magneto-optical disks, such as the hard disk or the removable media drive.

The Web server 1106 is a system that sends out Web pages containing electronic data files in response to Hypertext Transfer Protocol (HTTP) requests or similar protocol requests from remote browsers (i.e. browsers installed in the computing devices) or in response to similar requests made through a mobile app or mobile application of the present invention installed on a computing device. The web server 1106 can communicate with the mobile app of the present invention and/or with a web browser installed on computing devices 1000A, 1000B to provide the user interface required for the medication compliance and drug abuse prevention system.

As shown in FIG. 11, application server 1104 may communicate with the server data store 1112. Data store 1112 may be an in-memory database, for example and/or may include database persistent storage which may be a hard disk drive.

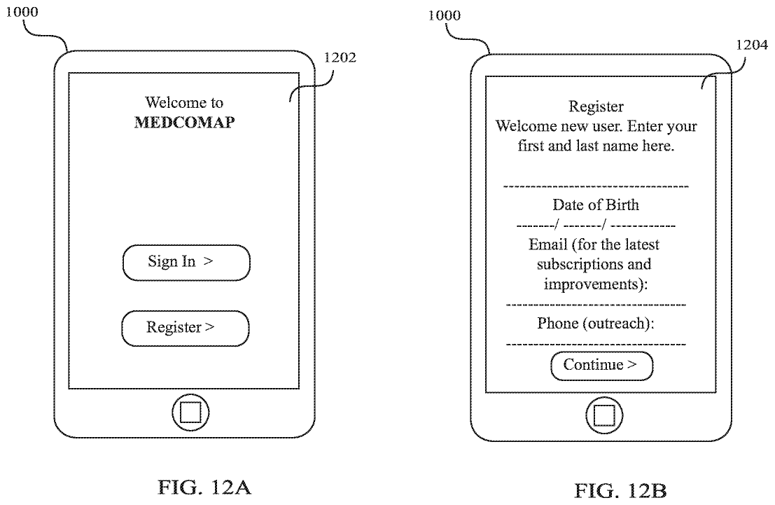

A user may sign-up with the MEDCOMAP system 1100 by providing required details. For this purpose, the user is required to access the user interface, as in step 1708 of FIG. 17, presented by the MEDCOMAP system server 1101, as in step 1706, and/or by the MEDCOMAP app 1006 on the computing device 1000. The user interfaces 1202 and 1204 presented on the computing device 1000, as shown in FIG. 12A and FIG. 12B, enable a new user to submit the required information for user registration or let an existing user to login as in step 1710.

For the implementation of the medication compliance and drug abuse prevention system of the present invention it is essential that the system has the required information on the medication prescribed. The information on medication includes one or more medication instructions/medication regimen/medication schedule, one or more data on the prescribed medicine comprising commercial name, chemical composition, weight/mass of each medicine/pill/tablet/capsule and total weight/mass of the medicinal substance supplied in a medicine container etc. The system can obtain such information using various options. For example, the prescription can be uploaded/scanned by using the user interface provided on the computing device 1000 and the information on medication can be obtained by the computing device or by the MEDCOMAP system server 1101 from the drug manufacturer and/from any third party database as in step 1712 of FIG. 17. The data is then stored locally at the medication compliance cap 200 or 300 and/or at the computing device 1000 and/or at the MEDCOMAP system server 1101 as in step 1714.

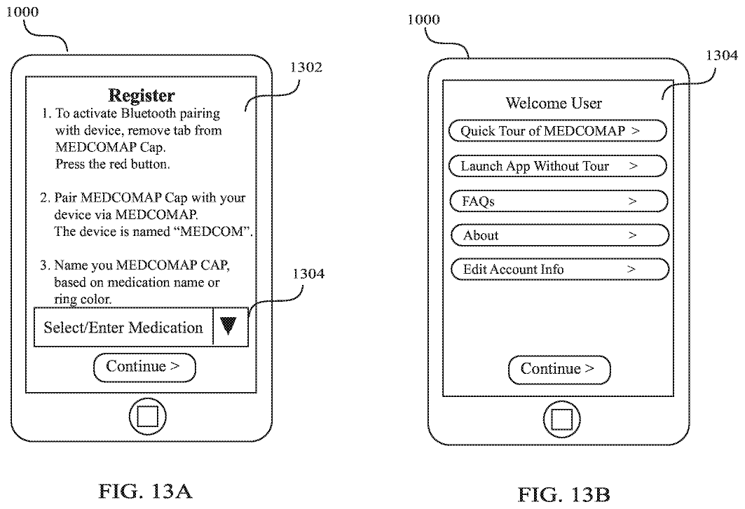

The user interface provides multiple controls with respect to the functioning of the medication compliance cap as in step 1716 of FIG. 17. Each medication compliance cap is assigned with a unique identity and the medication compliance caps can be associated with a particular user by this unique identity as in step 1718. This enables the computing device 1000 and/or the MEDCOMAP system server 1101 to identify each medication compliance cap associated with a user identity uniquely. The user interface 1302 presented on computing device 1000, as shown in FIG. 13A, helps the user to carry out this step. For example, a medication compliance cap can be associated with a user account by pairing the medication compliance cap with the computing device through Bluetooth following the steps shown on the user interface 1302. Each medication compliance cap can be given a name as per the user's convenience for easy identification. The user can then select or enter a medication to associate with the paired medication compliance cap using option 1304 of the user interface. A user can get more information on the MEDCOMAP system from a user interface 1304 as shown in FIG. 13B.

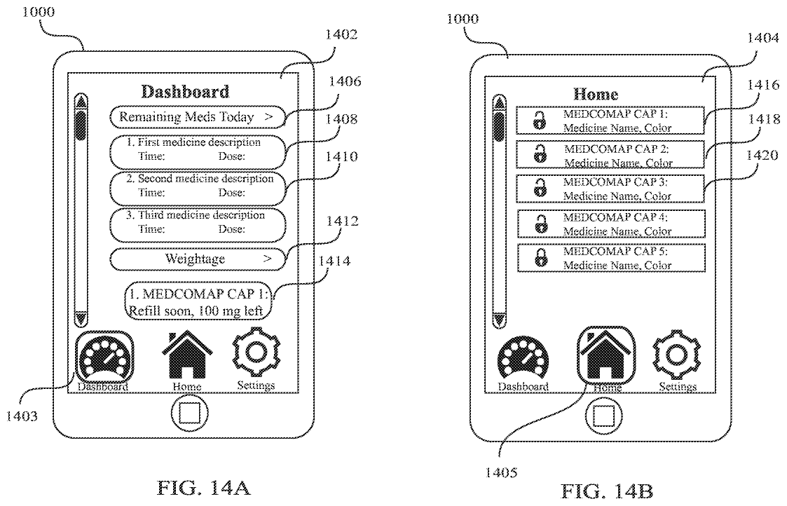

The computing device 1000 and/or the MEDCOMAP system server 1101 are configured to collate the data obtained from each medication compliance cap associated with a user and to present these data along with the medication instructions to the users as in step 1722 of FIG. 17. A user can get this information on the user interface, as in step 1724, by clicking on a particular button/icon presented on the user interface. For example, as shown in FIG. 14A, the dashboard screen 1402, displayed on clicking the dashboard button 1403, presents information 1406 on the medicines to be taken by the user for the day, information 1408 on description, time and dose for the first medicine, information 1410 on description, time and dose for the second medicine, information 1412 on weight/mass of a medicine and information 1414 on availability of a medicine in a medication compliance cap attached container etc.

For each medication compliance cap which has been attached to a container having a medicinal substance and is associated with a user identity/account, the timing of keeping the seal formed between the cap neck 204 or 304 and the container neck 106 or 112 in a locked state or in an unlocked state can be set as per requirement or as per the medication instructions. The lock/unlock status of all such associated medication compliance caps can be presented on the user interface. FIG. 14B illustrates such an exemplary screen 1404 with status 1416, 1418 and 1420 etc. of medication compliance caps under the home button 1405 on the user interface.

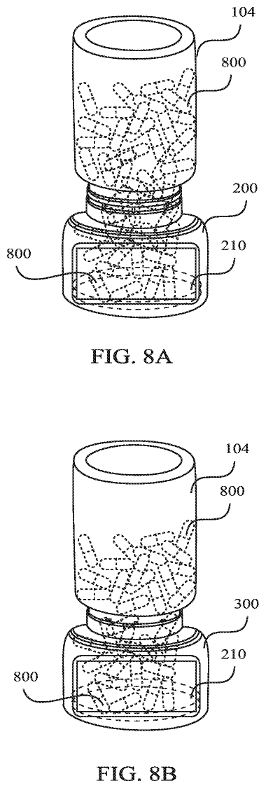

When the medication compliance cap attached to a container is kept vertically upright (with the container upside down) the medicine or the medicinal substance 800 kept inside the container flows through the passage to the cap internal compartment as shown in FIGS. 8A and 8B. In such a condition, the medicinal substance (capsule/pill/tablet or liquid) 800 rests on the weighment module 210 if the medication compliance cap and the weighment module 210 can measure a mass of the medicinal substance as in step 1726 of FIG. 17.

If a medicine/medicinal substance contained in a container attached to a medication compliance cap/MEDCOMAP cap, such as MEDCOMAP cap 1 (MEDCOMAP caps can be assigned any user preferred name for association with a user identity) is required to be taken by the user between 8 AM and 11 AM once daily as per medication instruction, then the MEDCOMAP cap 1 is configured to remain in an unlocked state during this period (referred to as second specific period of time) as in step 1728 of FIG. 17. In an unlocked state, the locking mechanism makes the teeth to withdraw and remain in a retracted position so that the medication compliance cap can be removed from the container to which it was attached. For the rest of the day (referred to as first specific period of time) the MEDCOMAP cap 1 remains in a locked state so that the MEDCOMAP cap 1 cannot be removed from the container to take out medicine from it.

In a preferred embodiment, even during the second specific period of time 8-11 AM in which the MEDCOMAP cap 1 should remain in unlocked state, the control module 214 is configured to let the locking mechanism 206 or 306 to disengage the lock only when a user authentication process is done. The user authentication process can be carried out in several ways—by verifying a user information such as biometric information of the user (or of the user authorized person/caregiver) taken through the computing device 1000 or through the built-in cap user interface 224 of the medication compliance cap, or by verifying a unique code (pre-generated code/password or a one-time password generated by the system) etc. The user authentication step ensures that only the intended user can access the medication contained in medication compliance cap attached container. The user authentication step also allows a user to access the medication in case of an emergency use beyond the specific unlock period.

A user can be given an audio/visual alert whenever a medication is due as per the medication instruction applicable to the user. For example, the light source 218 shown in FIGS. 2B-2C may indicate the unlock period by glowing in different colors. The speaker 216 may also give an audible alert. In addition, the computing device 1000 can also give such audio/visual alerts/notifications to the user.

The medication compliance cap is configured to measure the mass of the medicinal substance contained inside the container in a locked state after every time the medication compliance cap goes through an unlocked state. For example, in case of MEDCOMAP cap 1, it gets locked as in step 1730 after the unlock period of 8-11 AM as in step 1728 of FIG. 17. The mass of the medication taken during this locked state as in step 1732 is compared by the control module 214 locally or by the computing device/MEDCOMAP system server remotely to find the difference in mass of the medicinal substance measured before and after unlock state as in step 1734.

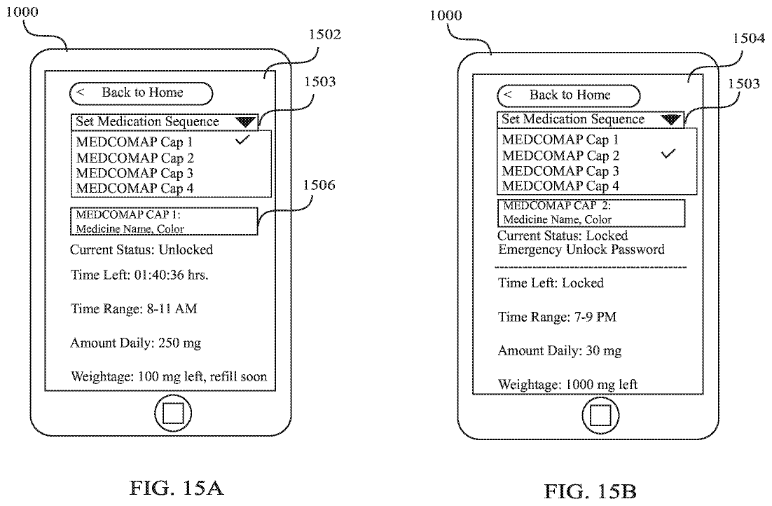

Ideally, the difference in mass of the medicinal substance taken before and after an unlock state should be equal to a prescribed mass of medication as determined from one or more medication instruction/information already available with the system. For example, as shown on user interface 1502 in FIG. 15A, during the time period of 8-11 AM, the user is required to take 250 mg of the medicinal substance from the MEDCOMAP cap 1 (medication name and color of the medication compliance cap 1 etc. can be shown in box 1506 on the user interface) attached container. If the difference in the mass of this medicinal substance measured before and after this unlock state period of 8-11 AM is found to be 250 mg then it is determined, as in step 1736, that the user has taken the recommended dose of medicine at the right time and the system updates the data as in step 1740 for future reference. If the difference in the mass of the medicinal substance measured before and after the unlock state is found to be less than or greater than the prescribed mass of medicine (250 mg is the prescribed mass of medicine in the present example) then it is determined to be an anomaly in step 1736 and an appropriate alert/preventive action is taken by the system as in step 1738.

If the difference in the mass of this medicinal substance measured before and after this unlock state is found less than the prescribed mass of medication then it is determined that the user has not taken the recommended dose of medicine. If the difference in the mass of this medicinal substance measured before and after this unlock state is found to be more than the prescribed mass of medication then it can be a case of drug abuse. For example, if instead of the prescribed dose of medication 250 mg, the difference in the mass of this medicinal substance measured before and after the unlock state is found to be 500 mg, then necessary alert/preventive actions are required to be taken against drug overuse or abuse. Therefore, as part of such an alert/preventive action, the medication compliance cap is configured to remain in a locked state, as in step 1742 of FIG. 17, until a manual override instruction is given to the control module 214 of FIG. 9. The system maintains a record of all the lock and unlock states and can present the same for audit/verification.

FIG. 15B illustrates an exemplary screen of user interface 1504 which depicts status of a second medication compliance cap, MEDCOMAP cap 2, associated with the same user. This screen shows the option of using emergency unlock password in case of a missed dose of medication.

In many cases a patient is required to take different medicines at different times of a day in a specific sequence as per their medication schedule/prescription. The MEDCOMAP system can guide a user to take the medication dosage in the required sequence by setting the lock and unlock states of multiple MEDCOMAP caps in a particular order of time periods. For example, if the medicinal substance from MEDCOMAP cap 1 enabled container is required to be taken before the medicinal substance from MEDCOMAP cap 2 enabled container, then the MEDCOMAP cap 2 will get unlocked only after MEDCOMAP cap 1 is unlocked and prescribed mass of medication is taken out of MEDCOMAP cap 1 enabled container. FIGS. 15A and 15B illustrate option 1503 on the user interface for setting the desired medication sequence with respect to the MEDCOMAP caps associated with the user identity/account. In case the user misses the medication from the MEDCOMAP cap 1 enabled container then the MEDCOMAP app may present on the user interface one or more instructions to suggest the user on what to do next rather than taking the medication from the MEDCOMAP cap 2 enabled container directly. Alternatives can include shutting off the entire sequence of medication until the next day or resetting the sequence such that regardless of the time of day, user is able to take medication from the MEDCOMAP cap 1 and then from MEDCOMAP cap 2 together. Other options may be suggested and keyed into the user's MEDCOMAP app based on their medical professional's advice.

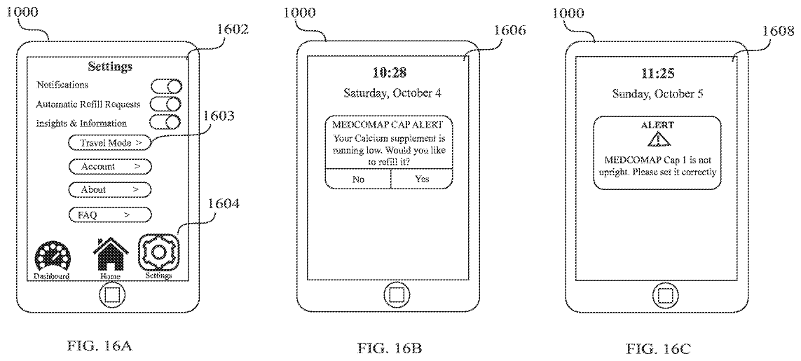

The user interface presented on the computing device can provide many other features such as notification on required refills and auto refill option etc. FIG. 16A illustrates such a screen of user interface 1602 viewable under the settings button 1604. A refill alert is depicted on user interface 1606 of FIG. 16B. All the features provided on the user interface of first computing device 1000A used by a patient/first user can also be provided to one or more other computing devices such as 1000B used by a caregiver or any such third person authorized by the first user.

In some embodiments, as shown in FIG. 9, the MEDCOMAP cap further comprises an orientation sensor 907 such as a gyroscope which can produce readings related to orientation data about three axes of rotation. The processor 902 is configured to read the orientation data and calculate specific movements of the MEDCOMAP cap about any axis. This enables the MEDCOMAP cap to detect its orientation and take appropriate action. For example, if the MEDCOMAP cap 1 is inadvertently kept upside down or horizontally or at any such position in which the weighment module 210 cannot measure the mass of the medicinal substance 800, then the MEDCOMAP cap 1 will give a visual and/or audible alert (user interface 1608 of FIG. 16C depicts one such alert) and keep the seal locked. Once the MEDCOMAP cap is kept in right position i.e. the top end 203 vertically above the bottom end 205, the MEDCOMAP cap will start functioning normally and validate the measurement of the mass of the medicinal substance 800.

In some embodiments, the MEDCOMAP cap is provided with a gesture detection module 905 as shown in FIG. 9. The gesture detection module comprises a radio frequency transmitter configured to emit radio frequency signals and a receiver configured to receive radio frequency signals reflected from an object. The processor 902 evaluates the transmitted and received signals and identifies a motion of the object. The gesture detection feature enables the MEDCOMAP cap to interact with a user in response to a gesture (hand gesture, for example) made by the user. For example, this feature may help users who need to keep their medication next to their beds/bedside and interact with the MEDCOMAP cap in the dark. A wave next to their bedside may be recognized by the MEDCOMAP cap as intent for the user to take the medication, assuming the time band of taking the dosage is correct. The wave or another similar such gesture may be recognized by the gesture detection module to turn the light source 218 on or make it brighter than the default level.

In another embodiment, MEDCOMAP system can collaborate with third party services to provide emergency notification/alert to users. With user's approval, the MEDCOMAP system may give third party services access to the user's medication information. One benefit of doing so besides centralizing all health information into the default app of the user's choice is the ability to read and process sensor data for medicinal use. Such third party services (which may be in the form of a mobile app, for example) may analyze the vital physiological parameters of the user on the basis of readings received from wearable sensors used by the user. Additionally, the MEDCOMAP system server 1101 and/or the MEDCOMAP app may also analyze such user health related data received from wearable sensors. If at any instance an emergency medical condition is detected and the user is required to take a medication on emergency basis then the applicable MEDCOMAP cap can be instructed to get unlocked, even if the MEDCOMAP cap is supposed to remain locked at that particular time as per its routine medication schedule, and an emergency visual/audible notification is triggered through the MEDCOMAP cap and/or MEDCOMAP app to prompt the user to take the appropriate medicine immediately. The consumption of another medicinal dose out of a time band or dosage metering in such cases is recorded and accounted for in subsequent measurement of the medicinal substance left in the container by the MEDCOMAP system.

Users anticipating travel may not want to carry one or more MEDCOMAP cap enabled pill bottles/containers along with them. For such cases, the MEDCOMAP system allows use of a travel mode provided in the MEDCOMAP app. User interface 1602 of FIG. 16A depicts an exemplary “Travel Mode” button 1603 for accessing this feature. On activating the Travel Mode feature, the user is allowed to enter the number of travel days for which the MEDCOMAP cap(s) will remain unused and the MEDCOMAP cap(s) will accordingly get unlocked to allow the user to remove required dose/mass of medicinal substance from the MEDCOMAP cap(s). The MEDCOMAP cap(s) will take into account the total mass of medicinal substance removed from the container(s) under the travel mode and check it against the prescribed medication for the number of travel days entered. The user is also allowed to return the medicinal substance previously removed under the travel mode to the containers after the end of travel days or before such date after making appropriate entry in the MEDCOMAP app.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they include structural elements that do not differ from the literal language of the claims, or if they include equivalent structural elements with insubstantial differences from the literal languages of the claims.

While the present invention is susceptible to various modifications and alternative user interfaces, a certain illustrated embodiment thereof is shown in the drawings and has been described above in detail. It should be understood, however, that there is no intention to limit the invention to the specific form or forms disclosed, but on the contrary, the intention is to cover all modifications, alternative user interfaces, similar features and equivalents falling within the spirit and scope of the invention, as defined in the appended claims.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. All methods described herein can be performed in any suitable order unless otherwise indicated herein or otherwise clearly contradicted by context. The use of any and all examples, or exemplary language (e.g., “such as”) provided herein, is intended merely to better illuminate embodiments of the invention and does not pose a limitation on the scope of the invention unless otherwise claimed. No language in the specification should be construed as indicating any non-claimed element as essential to the practice of the invention.

The flowchart is used to describe the steps of the present invention. While the various steps in the flowchart are presented and described sequentially, some or all of the steps may be executed in different orders, may be combined or omitted, and some or all of the steps may be executed in parallel. Further, in one or more of the embodiments of the invention, one or more of the steps described above may be omitted, repeated, and/or performed in a different order. In addition, additional steps, omitted in the flowcharts may be included in performing this method. Accordingly, the specific arrangement of steps shown in FIG. 17 should not be construed as limiting the scope of the invention.

What is claimed is:

1. A medication compliance and drug abuse prevention device for use as a cap with a medicine container, said device comprising:
a main body comprising a top end, a bottom end and a side wall extending between said top end and said bottom end, said side wall defining a cap internal compartment, wherein said cap internal compartment opens at said top end;
a cap neck disposed at said top end, said cap neck adapted for mating with a container neck of a container and forming a seal therewith, wherein said cap neck in association with said container neck defines a passage between said cap internal compartment and a container internal space of said container;
a locking mechanism disposed at said cap neck, said locking mechanism being capable of maintaining said seal in a locked state or in an unlocked state as per requirement;
a weighment module disposed inside said cap internal compartment, said weighment module configured to measure a mass of a medicinal substance contained inside said container internal space; and
a control module comprising a processor, said processor being configured to execute one or more instructions to:
configure said locking mechanism to maintain said seal in said locked state for a first specific period of time;
configure said locking mechanism to maintain said seal in said unlocked state for a second specific period of time;
determine a difference in said mass of said medicinal substance by comparing a first value of said mass of said medicinal substance measured in said locked state with a second value of said mass of said medicinal substance measured in said locked state after said unlocked state; and
enable said control module to initiate one or more alert actions if said difference in said mass of said medicinal substance is found to be less than or more than a prescribed mass of medicinal substance.

2. The device of claim 1, wherein said main body further comprises a built-in user interface.

3. The device of claim 1, wherein said locking mechanism comprises a collar, an actuator and one or more teeth.

4. The device of claim 3, wherein said collar is adapted to snugly fit around an unthreaded area of said container neck and said one or more teeth get engaged with said collar in said locked state.

5. The device of claim 1, wherein said first specific period of time and said second specific period of time are predetermined period of time as per one or more medication instructions for said medicinal substance and/or as per result of a user authentication process.

6. The device of claim 5, wherein said user authentication process includes successful verification of a unique code or a user information.

7. The device of claim 1, wherein said one or more alert actions initiated when said difference in said mass of said medicinal substance is found to be more than said prescribed mass of medicinal substance includes an action on maintaining said locked state until a manual override instruction is received by said control module.

8. The device of claim 1, wherein said one or more instructions are received from a computing device and/or from a remote server, said control module being communicatively connected to said computing device and/or to said remote server over a network.

9. The device of claim 1, wherein said one or more alert actions include an audible alarm emitted by a speaker disposed on said main body and/or a visual alarm emitted by a light source disposed on said main body.

10. A system for medication compliance and drug abuse prevention, said system comprising:
a medication compliance cap for use with a container, said medication compliance cap comprising a cap internal compartment, a cap neck adapted for mating with a container neck of said container and forming a seal therewith, a locking mechanism capable of locking said seal, a weighment module configured to measure a mass of a medicinal substance contained inside said container and a control module; and
a first computing device communicably connected to said medication compliance cap, said first computing device comprising one or more physical processors programmed with computer program instructions that, when executed by said one or more physical processors, program said first computing device to:
configure said locking mechanism to maintain said seal in a locked state or in an unlocked state as per requirement;
determine a difference in said mass of said medicinal substance measured before and after said unlocked state; and
initiate one or more alert actions if said difference in said mass of said medicinal substance is found to be less than or more than a prescribed mass of medicinal substance.

11. The system of claim 10, wherein said first computing device is further configured to identify said medication compliance cap by a unique identity.

12. The system of claim 11, wherein said first computing device is further configured to associate each of said unique identity of a plurality of said medication compliance caps with a user account of a user through a user interface provided on said first computing device.

13. The system of claim 11, wherein said first computing device is further configured to enable association of one or more data with respect to said medicinal substance with said unique identity of said medication compliance cap.

14. The system of claim 12, wherein said first computing device is further configured to enable presenting of one or more medication instructions with respect to said plurality of said medication compliance caps associated with said user account through said user interface of said first computing device.

15. The system of claim 14, wherein said first computing device is further configured to interact with a second computing device communicably connected with said first computing device for said one or more medication instructions and/or for said one or more alert actions.

16. The system of claim 10, wherein said one or more alert actions include keeping said locked state maintained until a manual override instruction is activated through said control module if said difference in said mass of said medicinal substance is found to be more than said prescribed mass of medicinal substance.

17. The system of claim 10, wherein said medication compliance cap is configured to detect an orientation of said medication compliance cap and initiate said one or more alert actions if said medication compliance cap is not kept in a right position.

18. The system of claim 12, wherein said medication compliance cap is configured to detect one or more gestures, made by said user, with the help of a gesture detection module included in said medication compliance cap.

19. The system of claim 12, wherein said unlock state of each of said plurality of said medication compliance caps is set in a specific sequence.

20. The system of claim 12, wherein said unlock state is activated and an emergency visual and/or audible notification is triggered on detection of an emergency medical condition of said user associated with said user account.

21. A method for medication compliance and drug abuse prevention, said method comprising:
providing a medication compliance cap for use with a container, said medication compliance cap comprising a cap internal compartment, a cap neck adapted for mating with a container neck of said container and forming a seal therewith, a locking mechanism capable of locking said seal, a weighment module configured to measure a mass of a medicinal substance contained inside said container and a control module;
configuring a first computing device to present a user interface on said first computing device, said first computing device being communicably connected to said medication compliance cap;
associating a unique identity of said medication compliance cap with a user account using said user interface;
associating one or more data with respect to said medicinal substance contained in said container with said unique identity of said medication compliance cap;
storing one or more medication instructions with respect to said medicinal substance;
configuring said locking mechanism to maintain said seal in a locked state or in an unlocked state as per said one or more medication instructions and/or as per a user authentication process;
determining a difference in a mass of said medicinal substance measured before and after said unlocked state; and
initiating one or more alert actions if said difference in said mass of said medicinal substance is found to be less than or more than a prescribed mass of medicinal substance as per said one or more medication instructions.

22. The method of claim 21, wherein said one or more alert actions include keeping said locked state maintained until a manual override instruction is activated through said control module if said difference in said mass of said medicinal substance is found to be more than said prescribed mass of medicinal substance.

23. The method of claim 21, wherein said user authentication process includes successful verification of a unique code or a user information.

24. The method of claim 21, wherein said first computing device is further configured to interact with a second computing device communicably connected with said first computing device for said one or more medication instructions and/or for said one or more alert actions.
